(19) United States
(12) Patent Application Publication
(10) Pub. No.: US 2008/0228175 A1
(43) Pub. Date: Sep. 18, 2008

(54) Medical device

(75) Inventors: Robert Snell, Suffolk (GB); Sam Martin, Suffolk (GB); Martin Cable, Cambridgeshire (GB); Eleanore King, Northants (GB)

Correspondence Address: WOLF GREENFIELD & SACKS, P.C., 600 ATLANTIC AVENUE, BOSTON, MA, 02210-2206, US

(73) Assignee: Ranier Limited, Cambridge (GB)

(21) Appl. No.: 12/075,658

(22) Filed: Mar. 13, 2008

Related U.S. Application Data
(63) Continuation of application No. 10/870,434, filed on Jun. 18, 2004, now abandoned.
(60) Provisional application No. 60/482,140, filed on Jun. 24, 2003.

(30) Foreign Application Priority Data
Jun. 20, 2003 (GB) 0314504.2
May 19, 2004 (DK) PA 2004 00806

(51) Int. Cl. A61M 27/00

(52) U.S. Cl. 604/544

(57) ABSTRACT

The invention provides a medical device with a retention section for anchoring the medical device in the body of a living being, a drainage section for draining fluids from the body, e.g. urine, and a tip which facilitates insertion into the body. During the anchoring of the medical device, the tip is retracted toward a proximal end of the device, improving the users control over the position of the tip relative to other parts of the device and achieving the free flow of fluids through the device. An indicator may be provided to give a visual, audible, or tactile indication of at least a first position of the tip relative to the remaining part of the medical device.

This application is a continuation of application Ser. No. 10/870,434 filed Jun. 18, 2004 entitled “Medical Device,” which, in turn, claims the benefit of U.S. provisional Ser. No. 60/482,140, filed Jun. 24, 2003.

### INTRODUCTION

The present invention relates to a medical device for draining fluids from the body of a living being. In particular, the invention relates to a medical device with a distal end for insertion into a body of a living being and an axially opposite proximal end, the device comprising: a tip which in a first position forms the distal end of the device, a drainage section forming a conduit extending in an axial direction of the device, and a retention section extending between the draining section and the tip, wherein the tip is operable between the first position and a second position so as to move the retention section between a first configuration and a second configuration, in which second configuration the retention section is expanded in a direction transverse to the axial direction. More specifically the present invention relates to a medical device comprising indicating means which indicates when the tip is in a predetermined posit ion when operated between the first and second position.

### BACKGROUND OF THE INVENTION

Many people suffer from a variety of medical problems that require the insertion of a medical device such as a catheter, to drain fluids from the body. For example, the most common treatment for problems with the urinary system, such as inability or difficulty in passing urine or severe incontinence, is to fit a catheter to drain urine from the bladder. A very well known catheter for this purpose is known as a Foley catheter. This is a flexible tube, usually made from latex or silicone, which has a central drainage lumen for the passage of urine and an inflation lumen communicating with an inflatable balloon incorporated into its distal end. With the balloon deflated, the catheter tube can be passed through the urethra into the bladder. Once the balloon is located in the bladder, it is inflated via the inflation lumen so that the balloon sits at the neck of the bladder and retains the tube in position. A tubular extension extends beyond the balloon into the bladder and has at least one eyelet in its wall through which urine may pass from the bladder into the central lumen. The urine flows down the tube to its proximal end situated outside the body which has a connector to which a collection bag is releasably fitted. The proximal end of the tube also has an inflation port for connection of a device to inflate the balloon via the inflation lumen.

Another type of conventional medical device comprises a tube having a retention section located at its distal end. The retention section has a cross-sectional diameter normally greater than the cross-sectional diameter of the remainder of the tube and of the urethra or other bodily conduit through which the medical device is to be inserted. To insert or remove a device of this type, an axial stretching force is applied to the distal end of the device using an insertion tool such as a rod or boogie temporarily inserted through the lumen of the device to engage with the retention section. This elongates or necks down the retention section so that its cross-sectional diameter reduces or stretches to a diameter similar to the remainder of the tube and less than that of the bodily conduit.

One such medical device is known from U.S. Pat. No. 5,041,093 which discloses a catheter comprising an axially and radially elastically extensible, foramenous woven tube having two ends disposed between the end of a tubular member and a tip, the tip being spaced from the tubular member. The woven tube is translatable between three configurations; relaxed, extended and over-center. In the over-center configuration the woven tube is doubled back on itself to form a cup- or disc-like shape. The woven tube may be arranged into a tulip-like shape which protectively encloses the tip. However in this configuration the opening of the tubular member is more or less blocked by the tip and thus it is desired to provide a solution wherein the tip is shielded by the over-center woven tube without blocking the opening of the tubular member.

Other examples of medical devices may be found in GB 0,766,308; GB 2,333,461; GB 1,463,269; GB 1,046,478; GB 1,014,570; GB 0,955,490; GB 688,450.

It is an object of a preferred embodiment of the present invention to provide a medical device facilitating better control over the anchoring mechanism.

### DESCRIPTION OF THE INVENTION

Accordingly, the present invention relates to a medical device with a distal end for insertion into a body of a living being and an axially opposite proximal end, the device comprising: a tip which in a first position forms the distal end of the device, a drainage section forming a conduit extending in an axial direction of the device, and a retention section extending between the draining section and the tip, wherein the tip is operable between the first position and a second position so as to move the retention section between a first configuration and a second configuration, in which second configuration the retention section is expanded in a direction transverse to the axial direction.

It one embodiment the medical device comprises indicating means which indicates when the tip is in a predetermined position when operated between the first and second position.

An advantage of the present invention is that it is possible for the user to remote control the position of the tip e.g. such that the tip has been retracted exactly enough to make sure that it can not irritate the inner wall of a body lumen e.g. a bladder as a retention section will partly or completely shield the tip.

In some of the passages in the remaining part of the description, the medical device is described with reference to catheters to be inserted into a urinary drainage, i.e. wherein the catheter is inserted into a natural or artificial urinary canal e.g. urethra, and into a bladder for draining urine during use, the catheter could be inserted into a body opening and guided into a cavity to be drained for body fluid. The medical device could, however, be used for draining body fluids in general, be that either subcutaneously or through insertion of the medical device into a natural or artificial opening in the body, or the medical device could be applied for stent delivery, e.g. for placing a stent within the prostatic urethra, or in general for draining fluids from a natural or artificial body lumen, for anal insertion or for insertion into the gastric region. Another type of use could be for intubations or endoscopy.

The indicating means may be provided as a visible, audible or tactile signal. The visible signal may be a coloured area or a symbol provided in the proximal end at a position which extends out of the body when the medical device is inserted into the body. Furthermore the visible signal could be a lines or scales provided on a deployment member. An audible or tactile signal could be generated by two parts which during relative movement engage or disengage each other and thereby generates sound or tactile indication. The indicating means may be provided in the proximal end or at any other part of the device.

By providing indication means which indicates the position of the tip of the medical device in relation to the retention section it is possible for the user to insert the medical device into the body with the tip in the first position, and to anchor the medical device in the body by retracting the tip to the second position. During the movement of the tip between the positions, the user obtains a signal indicating the position of the tip and thereby obtains improved control over the use of the medical device. The indicating means may further be adapted to signal when the tip passes second, third or further positions during the movement, e.g. one position when the tip is retracted not to form the distal end, one position wherein the tip is in the area of the conduit but still not retracted into the conduit, and one position wherein the tip is located in the conduit and thereby possibly blocks fluid passage.

The indicating means could form part of a deployment member or form part of a deployment mechanism provided in the proximal end of the medical device for controlling the deployment member. As an example, the deployment member could be constituted by an axially incompressible rod fastened between the tip of the device and the proximal end of the medical device, and the axial displacement of the deployment member could be controlled via a deployment mechanism comprising means allowing axial elongation or compression of the proximal part of the medical device. For this purpose, the medical device could have a corrugated proximal end portion forming bellows and including visually detectable means indicating the position of the tip in relation the first and optionally other positions.

In order to allow the user to lock the medical device in the first and optionally further positions of the tip, the deployment member or the deployment mechanism could comprise locking means for locking further movement of the tip. The locking means may be reversible i.e., adapted to be locked and un-locked.

In order to further increase the degree of expansion or to form a specific shape of the braided portion in the second configuration, a first part of the braided portion may be located inside a second part of the braided portion when the device is in the second configuration. The first and second parts of the braided portion does not have to be structurally separated, but could form one uniform braided portion, and merely the fold arising by the inverting or rolling of one part of the braided portion into another part of the braided portion defines the transition between the first and second parts of the braided portion. In the first configuration, the braided portion could extend un-folded in the axial direction.

The medical device, especially the retention section, may be designed with “shape-memory” such that it will automatically move towards a predetermined shape i.e. towards a relaxed state. In a first embodiment the medical device is designed such that the predetermined shape is the first configuration, i.e. the medical device will have a tendency to move towards the first configuration, but may be moved into the second configuration by axial displacement of the first part of the braided portion into a second part of the braided portion. In some embodiments the first configuration is a configuration wherein the largest dimension of a cross-section of the retention section is equal or less than the largest dimension of a cross-section of the drainage section.

In a second embodiment the medical device is designed such that the predetermined shape is the second configuration, i.e. the medical device will have a tendency to move towards the second configuration, but may be moved into the first configuration by axial displacement of the first part of the braided portion out of the second part of the braided portion. In some embodiments the second configuration is a configuration wherein the largest dimension of a cross-section of the retention section is larger than the largest dimension of a cross-section of the drainage section.

In a third embodiment the medical device is designed such that the predetermined shape when the retention section is located inside the remaining part of the medical device, e.g. the drainage section, coaxially therewith. When the medical device is located in the body, the second part is displaced out of the remaining part of the medical device to form a medical device in the second configuration, i.e. retained in the body. To operate the medical device between the different configurations, a deployment member could be fastened in the distal end, e.g. to a distal tip of the medical device, and extend to the proximal end to facilitate manipulation of the distal end from outside the body. To facilitate comfortable insertion or to reduce adherence of body tissue to the surface of the medical device, at least a part of the medical device, e.g. the braided portion or the second part of the braided portion or the tip-part may have an outer surface, i.e. a surface towards the body tissue when the medical device is inserted into the body, which surface has a low surface friction characteristic compared to other parts of the medical device. To provide the low friction characteristic, the braided portion or at least the second part thereof may have a hydrophilic surface, e.g. provided by a hydrophilic coating of the surface. A hydrophilic coating may further reduce irritation of the body tissue, e.g. mucosa. If a hydrophilic coating is applied to the braided portion, the coating may incorporate an anti-infective compound or a compound which counteracts ingrowth.

The retention section may comprise a braided portion with crossed filaments which mutually form a braiding angle. The braiding angle may vary along the length of the retention section e.g. such that the braiding angle in the middle of the retention section is different from the braiding angle in the top and bottom of the retention section.

The wording “braided portion” includes in general a device portion provided with through-going windows, i.e. openings formed from an outer peripheral surface to an inner peripheral surface and often being symmetrically arranged to form a uniform grid of windows. More specifically, the braided portion may comprise cross-braided filaments, i.e. threads which are braided over and under each other. Preferably, the braiding enables the filaments to slide relative to each other. Alternatively, the filaments are arranged in two separate and parallel layers wherein the filaments of one of the layers extend in a direction different from the direction of the filaments of the other layer. In each intersection between a filament of one of the layers and a filament of another layer, the filaments of the two layers may be joined by adhesion. The braided portion could also be constituted by a section of the device with openings forming a mesh-pattern. Irrespective of the type of braiding, the angle, α, which the filaments form with the axial direction, is important for determining the degree of radial expansion and the more precise shape of the retention section which arises when the first part is displaced into the second part of the braided portion. This is described in further details later.

The braiding may be provided in the retention section and/or the drainage section and/or the deployment member. Furthermore the tip of the device may be made of the same braided material as the retention section. In order to provide a homogeneous surface of the tip, the braided material may be shaped by means of a heated tool or a tool for plastically deforming the material in to e.g. a bullet-like shape.

By providing a drainage section comprising a braided material, the strength of the drainage section is reinforced as the braided section and an appropriate matrix material constitutes a composite. Accordingly it is possible to make the wall of the drainage section thinner. Thus for a given outer diameter of the medical device the cross-sectional flow area may be larger.

In the table below, it is indicated how the use of a braided drainage section can increase the cross section of the conduit in a catheter.

| | Traditional silicone | Cross sectional area |
|---|---|---|
| | catheter cross | with a braided |
| Ch size | sectional area [mm²] | drainage section [mm²] |
| 8 | 1.3 | 2.3 |
| 10 | 3.1 | 4.2 |
| 12 | 3.8 | 7.1 |
| 14 | 4.9 | 10.8 |
| 16 | 3.8 | 14.5 |
| 18 | 8.0 | 19.6 |

Moreover the filaments of the drainage section may form an extension of the filaments of the retention section.

At least the drainage section may be at least partially coated with polymeric material. Furthermore the retention section may be at least partially coated with polymeric material, the coating on the retention section being thinner or having the same thickness as the coating on the drainage section.

The filaments could e.g. be made from polyester, polyamide, polyalkane, polyurethane, PET, PBT, Nylon, PEEK, PE, Glass Fibre, Metal Wire or Acrylic materials or any composition of the mentioned materials. A preferred material would be PET or polyester.

The medical device may include a matrix material, e.g. any medical grade polymer that can be dissolved in a solvent or manufactured as a polymer emulsion. Examples of these are polyurethane, polyurethane dispersions, acrylic, PVC, block copolymers (SIS SBS) etc, natural rubber, silicone, neoprene, nitrile or compositions thereof. Polyurethane, acrylic, PVC, block copolymers (SIS SBS) etc, natural rubber, silicone, or EPO or compositions thereof, could be used if the medical device is made by extrusion or injection moulding.

In a second aspect the present invention relates to the use of a braided material for increasing the cross-section of a conduit throughout a medical device. By providing a braided material in a drainage section defining a conduit, it is possible to provide a drainage section which is has a larger cross-section for a given outer diameter. The filaments of the braiding in the wall of the braided section reinforces said wall and thus it may be thinner in order to provide the same strength.

### DETAILED DESCRIPTION OF THE INVENTION

Embodiments of the invention will now be described, by way of example only, with reference to the accompanying drawings, in which:

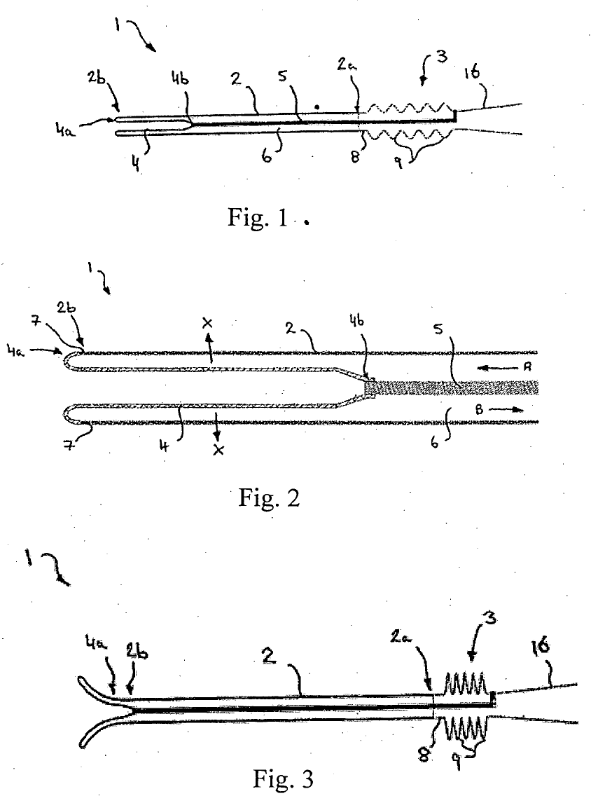

FIG. 1 illustrates a longitudinal cross-sectional view of the medical device with the retention section in a first configuration and ready for insertion into, or removal from, the patient;

FIG. 2 illustrates an enlarged view of the distal end portion of the medical device shown in FIG. 1;

FIG. 3 illustrates the medical device shown in FIGS. 1 and 2 in a second configuration in which the retention section has been deployed to retain the medical device in position in the patient;

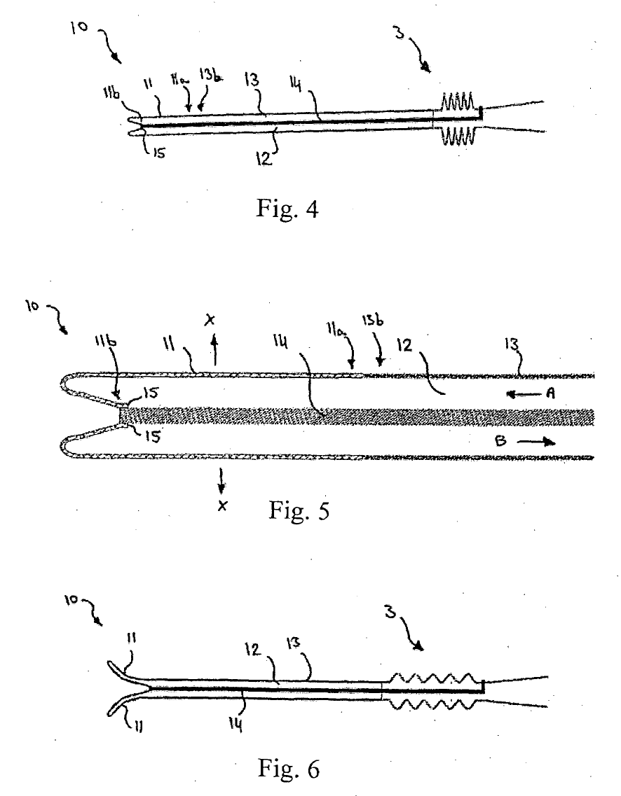

FIG. 4 illustrates a longitudinal cross-sectional view of a second embodiment in which the retention section is in a first extended configuration and is ready for insertion into, or removal from the patient;

FIG. 5 illustrates an enlarged view of the distal end portion of the medical device shown in FIG. 4;

FIG. 6 illustrates the medical device shown in FIGS. 4 and 5 in a second configuration in which the retention section has been deployed to retain the medical device in position in the patient;

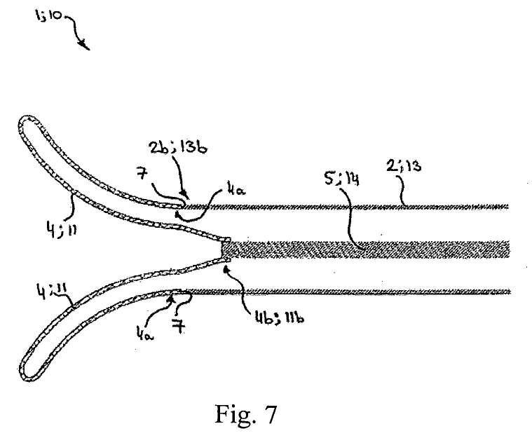

FIG. 7 illustrates an enlarged view of the distal end of the medical device shown in the first embodiment of FIG. 3 or the second embodiment of FIG. 6;

FIG. 8 illustrates a longitudinal cross-sectional view of the distal end of a third embodiment in which the retention section is in a first configuration and is ready for insertion into, or removal from, the patient;

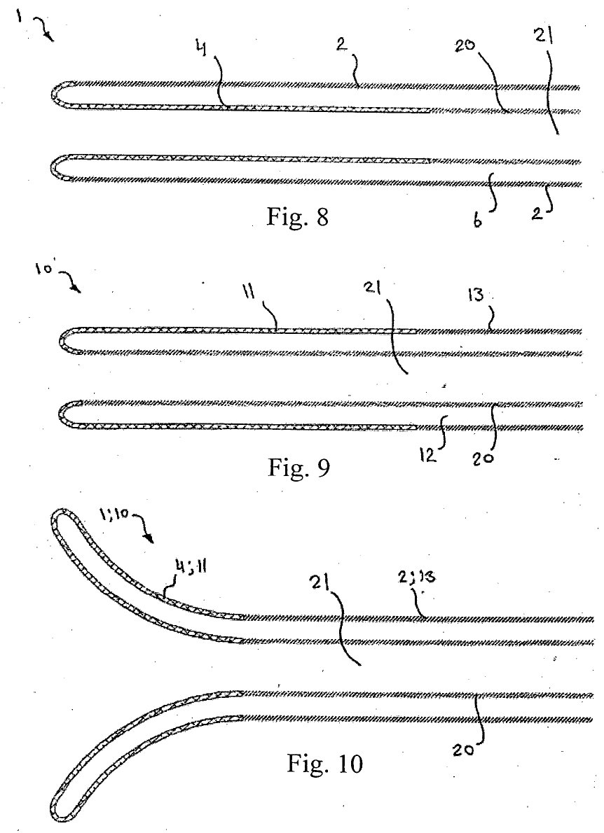

FIG. 9 illustrates a longitudinal cross-sectional view of the distal end of a fourth embodiment in which the retention section is in a first configuration and is ready for insertion into, or removal from, the patient;

FIG. 10 illustrates the distal end of the medical device of the third or fourth embodiment in which the retention section has been deployed into a second configuration to retain the medical device in position in the patient.

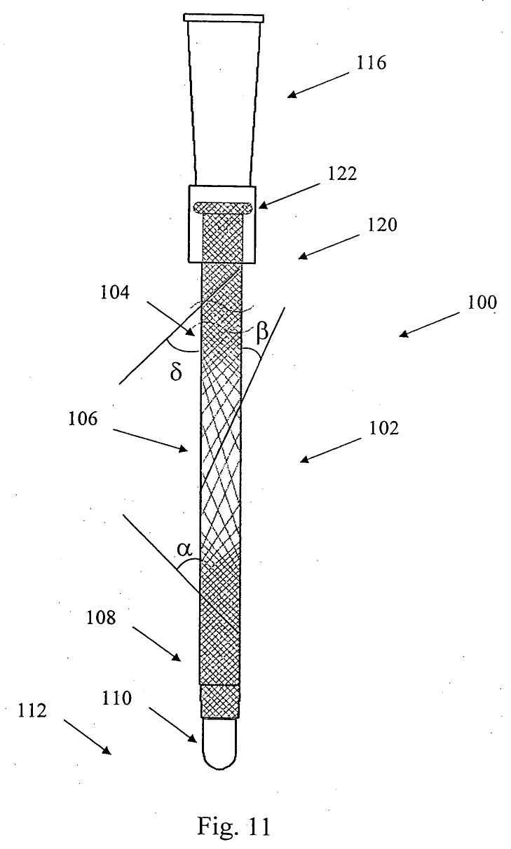

FIGS. 11-16 show the process of inverting or rolling over a braided retention section.

Referring now to the drawings, there is shown in FIG. 1 a medical device 1, e.g. a catheter, according to an embodiment of the invention in a first configuration in which it is ready for insertion into a patient, or removal therefrom. If the medical device is a urinary catheter it may be urethral such that it may be inserted through the urethra into the bladder of the patient or suprapubic in which case it is passed surgically into the bladder through a surgical incision in the abdominal muscle wall. Alternatively the medical device may be for intubation and thus be inserted through the esophagus. In other embodiments the medical device is for anal insertion.

The medical device 1 comprises an elongate flexible tubular resilient drainage section 2 having a proximal end 2a connected to a deployment mechanism 3 and a distal end 2b connected to one end 4a of a flexible tubular resilient retention section 4. The drainage section 2 has a lumen 6 for the passage of bodily fluids therethrough and is of a length to enable it to extend from the cavity or bladder to be drained out of the patient so that its proximal end 2a can be connected to a collection bag or other receptacle via the deployment mechanism 3. The other end 4b of the retention section 4 is connected to one end of a flexible resilient deployment member or rod 5 which is slideable axially in the direction indicated by arrow “A” (see FIG. 2) within the lumen 6 of the drainage section in response to operation of the deployment mechanism 3 to which the other end of the deployment member 5 is attached.

It should be noted that, throughout the specification, reference to the distal end refers to the end of the medical device or part of the medical device which is inserted deeper into the body of the patient and reference to the proximal end refers to the end of the medical device or part of the medical device which is closer to the end of the medical device which remains outside the body of the patient.

As can be seen more clearly from FIG. 2, which illustrates an enlarged partial view of the distal end of the medical device 1, the retention section 4 is held in a generally cylindrical shape and is almost entirely withdrawn into the lumen of the drainage section, in its first configuration. The end 4a of the retention section emerges from the lumen of the drainage section and is rolled over, inverted, invaginated or doubled back over itself, to connect the very tip 7 of the end 4a of the retention section to the distal end 2b of the drainage section for reasons that will become apparent. The opposite end 4b of the retention section 4 converges and is attached to the end of the rod 5 lying within the lumen 6 of the drainage section 2.

The retention section 4 is made from braided material formed from structural filaments that are cross-braided over and under each other. The braiding of the filaments is sufficiently spaced so that fluid may pass through spaces between the filaments and into the lumen 6 when the retention section 4 is in the second configuration. The braiding is also spaced enough to enable the interwoven strands or filaments to slide relative to each other so that the shape of the retention section 4 changes as it moves between the first and second configurations when forces are applied to it, as will be explained in more detail below.

The deployment mechanism 3 comprises a tubular component 8 attached to the proximal end 2a of the drainage section 2 so that the lumen 6 is coaxial with the tube 8. A portion 9 of the wall of the tube 8 is corrugated or is in the form of bellows that enable the tubular component 8 to be axially compressed to reduce its length from the extended non-compressed state shown in FIG. 1 to the compressed state shown in FIG. 3 and then returned to its original state shown in FIG. 1. The rod 5 passes through the corrugated portion 9 and is connected to the tube 8 so that as the corrugated portion 9 is compressed and extended, the rod 5 moves axially within the lumen 6 of the drainage section in the direction indicated by arrows “A” and “B” (see FIG. 2) respectively. The other end 16 of the tube 8 remote from the drainage section 2 widens into a larger diameter and may be provided with means thereon (not shown) for connection to a collection bag via a tube.

FIG. 3 shows the medical device 1 of FIGS. 1 and 2 when the retention section 4 has been moved into its second configuration after the device has been positioned within the patient's body and the retention section has been deployed to retain or anchor the catheter 1 in position by operating the deployment mechanism 3 to cause the rod 5 to move axially in the direction of arrow “A”. As can be seen most clearly from the enlarged partial view of the distal end of the catheter 1 in FIG. 7, the retention section 4 is pushed out of the distal end 2b of the drainage section 2 and forms the double-walled funnel shaped configuration due to the nature of the braided material from which the retention section is formed. The funnel shape of the retention section 4 in the second configuration prevents withdrawal of the medical device 1 from the patient.

To remove the medical device 1 from the patient, the retention section is retracted or withdrawn into the distal end of the drainage section by applying an axial force to the corrugated portion of the deployment mechanism so that the rod 5 now moves in the opposite direction as indicated by arrow “B”. Once the retention section 4 has been retracted or withdrawn, the medical device 1 can be removed from the patient.

The rolled end 4a of the retention section 4, described in more detail above, significantly assists the ease by which the retention section 4 is deployed from the distal end 2b of the drainage section 2. When the deployment member 5 moves in the direction of arrow “A”, the retention section 4 does not expand or bulge radially against the wall of the lumen of the drainage section 2 in the direction indicated by arrow “X” (see FIG. 2). Furthermore, the retention section 4 does not fold up inside itself within the part of the retention section 5 pressed against the wall of the lumen 6 before the rod 5 emerges from the distal end 2b of the drainage section 2, pulling the retention section 4 out of the distal end 2b of the drainage section 2 with it. On the contrary, the cross-sectional diameter of the retention section 4 remains substantially constant whilst it remains within the distal end 2b of the drainage section 2 and only expands in cross-sectional diameter as it emerges from the distal end 2b of the drainage section 2 to form the funnel or cup like shape illustrated in FIG. 3. Furthermore, the retention section 4 does not collapse or fold in on itself as the axial force is exerted against it by the rod 5. This is due to the rolled end 4a of the retention section in its first configuration which causes the retention section to continue to roll round the inverted end so as to become further inverted or doubled over to a greater extent because the force required to cause the retention section to bulge radially against the inner wall of the lumen is greater than the force required for the retention section to continue rolling round the inverted end 4a. Therefore, the retention section 4 remains generally tubular in shape within the distal end of the drainage section 2b and tracks or feeds round the rolled edge of the inverted end 4a to form the second configuration illustrated in FIG. 3 in which the retention section is substantially rolled in half, i.e. half the retention section 4 overlies the other half of the retention section 4.

It will be appreciated that the rolled end 4a of the retention section 4 in the first configuration is advantageous to assist in deployment of the retention section 4. However, the rolled end is not essential in this embodiment of the invention. In another unillustrated embodiment, the retention section may not be inverted at its end 4a in the first configuration. In this arrangement, the retention section 4 can still be deployed from the end of the drainage section into the second configuration illustrated in FIG. 3, but the retention section will first bulge radially and press against the inner wall of the lumen and then the retention section will pass up through itself as the rod 5 moves in the direction of arrow “A”. It will be appreciated that, in this embodiment, the end 4a of the retention section need not emerge from the distal end of the drainage section 2 and can be fully withdrawn into the drainage section in the first configuration.

A second embodiment will now be described with reference to FIGS. 4 to 6. FIG. 4 shows a medical device 10 similar to medical device 1 of FIG. 1 in which the retention section 11 is generally cylindrical in shape in the first configuration. However, instead of being withdrawn or retracted into the lumen 12 of the drainage section 13, the retention section 11 extends coaxially beyond the end of it and the deployment member 14 extends through the retention section 11 for connection to its second end 11b. As the cross-sectional diameter of the retention section 11 is generally less than or the same as the cross-sectional diameter of the drainage section 13, the medical device 10 can be inserted into or removed from the patient when it is in this first configuration.

In this embodiment, the second end 11b of the retention section 11 is rolled over, inverted or doubled back inside itself, to connect the very tip 15 of the end 11b of the retention section 11 to the end of the deployment member 14 e.g. a rod, as most clearly shown in FIG. 5 and for reasons that will become apparent. The opposite end 11a of the retention section 11 is attached to the distal end 13b of the drainage section 13.

FIG. 6 shows the medical device 10 of FIGS. 4 and 5 when the retention section 4 has been moved into its second configuration after the medical device has been positioned within the patient's body and the retention section has been deployed to retain the medical device 10 in position by operating the deployment mechanism 3 to cause the deployment member 14 to move axially in the direction of arrow “B”. As can be seen most clearly from the enlarged partial view of the distal end of the medical device 1,10 in FIG. 7, the end 11b of the retention section 11 is pulled by the rod 14 so that more of it is inverted or folded into itself to form the double-walled funnel shaped configuration, due to the nature of the braided material from which the retention section is formed. The funnel shape of the retention section 11 in the second configuration prevents withdrawal of the medical device 10 from the patient.

To remove the medical device 10 from the patient, the rod 5 is pushed in the direction of arrow “A” so that the retention section 4 returns to the position shown in FIG. 5.

The rolled end 11b of the retention section 11, described in more detail above, assists the ease by which the retention section 11 moves from the first to the second configuration when the end 11b of the retention section 11 is pulled by applying an axial force to the deployment member 14 in the direction of the arrow “B”. Instead of bulging or expanding radially outwardly in the direction indicated by arrow “X” before a continued application of the pulling force on the retention section 11 causes the end 11b to flip inside out or over-centre to form the double walled funnel-like shape, the rolled end 11b causes the retention section 11 to continue to roll, track or feed round the inverted end 11b so as to become further inverted or doubled over to a greater extent with limited or no initial bulging in a radial direction. Therefore, the funnel is formed gradually and in a controlled manner as the rod 5 is pulled. As the force required to cause the retention section to bulge is greater than the force required for the retention section to continue rolling round the inverted end 4b, the retention section 11 remains generally tubular in shape and tracks round the rolled edge of the inverted end 11b to form the second configuration illustrated in FIG. 3 in which the retention section is substantially rolled in half, i.e. half the retention section 11 overlies the other half of the retention section 11.

It will be appreciated that the first and second embodiments can be combined into one medical device in which the second configuration is an intermediate position between two optional first configurations in which the rod 5,14 is pulled to withdraw the retention section 4,11 into the lumen 6,12 of the drainage section 2,13, as described with reference to the first embodiment or, the rod 5,14 is pushed to cause the retention section 4,11 to elongate or extend beyond the distal end 2b,11b of the drainage section 2,13, as described with reference to the second embodiment.

It will be apparent that the deployment member 5,14 remains within the lumen 6,12 of the drainage section 2,13 and is not removed therefrom once the medical device 1,10 is in position. The deployment member 5,14 may therefore be integrally formed with the retention section 4,11 or may be made separately and permanently connected thereto by, for example, welding. To ensure free passage of fluid through the lumen 6,12 of the drainage section 2,13 the diameter of the rod 5,14 is much smaller than the diameter of the lumen 6,12 so that fluid can pass freely down through the lumen 6,12 around the rod 5,14. As the rod 5,14 remains in the lumen 6,12, insertion and removal of the medical device 1,10 is simplified.

Another embodiment is illustrated in FIG. 8 which is identical to the embodiment of FIG. 1 except that the deployment member 5 is a tube 20 which has a cross sectional outer diameter only slightly less than the cross sectional diameter of the lumen 6 of the drainage section 2 so that it can slide in an axial direction within the lumen 6. In this case, bodily fluids pass down through a lumen 21 in the tube 20 rather than through the lumen 6 of the drainage section 2.

The embodiment of FIG. 9 is identical to the embodiment of FIG. 4 except that the deployment member 14 is a tube 20, as explained with reference to the embodiment of FIG. 8.

FIG. 10 shows the medical device 1,10 of FIGS. 8 and 9 when the retention section 4,11 has been deployed into the second configuration.

Again, it will be appreciated that the embodiments of FIGS. 8 and 9 can be combined to form a medical device in which the second configuration illustrated in FIG. 10 is an intermediate position between optional first configurations illustrated in FIGS. 8 and 9 respectively.

The drainage section 2,13, the retention section 4,11 and the deployment member 5,14 of the medical device 1,10 of any of the embodiments of the invention may be manufactured as a single integral entity or continuum of material. Each section can be formed from braided material and changes in braid angle can be used, for example, between the drainage section 2,13 and the retention section 4,11 to control the degree by which these sections may vary in their cross-sectional diameter as an axial force is applied by the deployment member 5,14.

The deployment member 5,14 may be manufactured separately and permanently connected to the retention section 4,11 and/or the deployment mechanism 3 or it may be integrally formed with the retention section 4,11 and/or the deployment mechanism 3. For example, the monofilament braids of the retention section may be grouped or reduced in number to form the deployment member 5,14 and so be formed from a continuum of the retention section 4,11.

The deployment mechanism 3 may be integrally formed with the drainage section 2,13 or be fabricated as a separate component which is later removably or permanently joined thereto. The corrugated portion 9 may also be partially or wholly fabricated separately from the remainder of the tube 8 by, for example, injection or blow moulding. For example, the corrugated portion 9 may be formed separately to or integral with the end part 16 of the tube 8. Alternatively, the corrugated portion may be formed separately to the part of the tube 8 that connects it to the drainage section.

A locking device may be incorporated with the deployment mechanism 3 to maintain the corrugated portion 9 in either position and so prevent relative movement of the deployment member 5,14 with respect to the drainage section 2,13 until it is released. The relaxed state of the corrugated section is preferably as shown in FIGS. 3 and 4 and so it may not be necessary to provide a locking mechanism to hold the corrugated portion in the collapsed states shown in these figures. However, it is desirable to provide a locking mechanism to prevent the corrugated portion 9 from collapsing back into the configuration shown in FIGS. 3 and 4 when it has been axially stretched into the configuration shown on FIGS. 1 and 6 and subsequently released. It should be understood that the locking mechanism should be reversible i.e. the locking mechanism be able to lock and un-lock.

It will be appreciated that the medical device 1,10 is not limited for use with the deployment mechanism 3 described and that other types of deployment mechanism may be used to move the deployment member 5,14 relative to the drainage section 2,13.

The medical device 1,10 of the invention is made from a bio-compatible polymer material which may be polyurethane coated. However, in a preferred embodiment, the retention section 4,11 is not coated so that the braids remain open and relative movement between the braids is enabled. It is also envisaged that there could be a thinner coating on the retention section 4,11 but which is not so thick so as to prevent relative movement between the braids relative to each other. The retention section 4,11 and the drainage section 2,13 could both be coated during the manufacturing process and areas of the coating can subsequently be removed by, for example, ablation to improve the compliancy of those areas. The braiding of the drainage section may or may not be coated and/or instead the braiding may be welded at overlapping points to modify the degree of compliancy. Similarly, some of the overlapping points in the braiding of the retention section may be welded to modify the degree of compliancy of the retention section.

The retention section 4,11 may be formed from a non-braided material which acts in the same way as the braided material and will expand as it moves into the second configuration. However, a braided material is preferred and the filaments can be made from a number of different materials such as metallic wires, polypropylene, nylon, polyurethane or polyethylene.

The drainage section 2,13 may also be made from the same braided material as the retention section 4,11 including structural filaments that are arranged at an angle such that the cross-sectional diameter of the drainage section 2,13 decreases when an axial force is applied to the retention section 4,11 via the deployment member 5,14 but returns to its original diameter when the force is removed. However, the drainage section 2,13 can be made from braided material which is coated so that movement of the filaments relative to each other is restricted. The use of a braided structure increases the strength of the medical device 1,10 and its kink resistance. Furthermore, because there is no inflation lumen, an increase in the diameter of the lumen 6,12 is enabled and allows higher flow rates through the medical device 1,10.

Alternatively, the drainage section 2,13 can be fabricated from thin walled elastomeric tubing such as polyurethane, neoprene, styrene ethylene butadiene styrene (SEBS), styrene butadiene (SBS), plasticised PVC or thermoplastic vulcanites.

Many modifications and variations of the invention falling within the terms of the following claims will be apparent to those skilled in the art and the foregoing description should be regarded as a description of the preferred embodiments only.

In the following the FIGS. 11-16 are described in detail. The figs. show the process of inverting, rolling over, invaginating, or doubling back the retention section of a medical device of an embodiment of the present invention.

In FIG. 11 the medical device 100 is in a stretched state wherein it is possible to insert the medical device into a body of a living being e.g. into a bladder through the urethra. The medical device 100 comprises a braided retention section 102 comprising a bottom part 104, a middle part 106 and a top part 108. As may be seen from the figure the braiding angle of the filaments is different in the middle part 106 than in the bottom and top part 104, 108. The braiding has a first angle, α, in the top part 108, a second angle, β, in the middle part 106 and a third angle, δ, bottom part 104. In the disclosed embodiment, α equals δ

Furthermore the medical device 100 is provided with a tip 110 at the distal end 112 for facilitating insertion of the medical device. At the proximal end 114 there is provided a connector 116 adapted to be connected to e.g. a drainage bag (not shown).

A slidable coupling 188 with tactile indication of its position is provided between the connector 116 and a drainage section 120. The slidable coupling 118 comprises a corrugation 122, provided on an outer surface of the drainage section, and a first and second recess 124,126. The corrugation 122 is adapted to engage the first recess 124 and the second recess 126 and to slide between said two recesses. One advantage of such a slidable coupling is that it makes it possible to provide a compact medical device which prior to use is provided in a compacted state and may be extended into an operable state when the medical device is to be used. In the compacted state the corrugation 122 engages the first recess 124 and in the operable state the corrugation engages the second recess 126. A further advantage is that the user may sense e.g. in the fingertips when the corrugation is brought into or out of engagement with one of the recesses. This may be used to signal a predetermined relative position of the tip to the user. This is useful as the user in the use situation is not able to see the tip as it is provided inside the body.

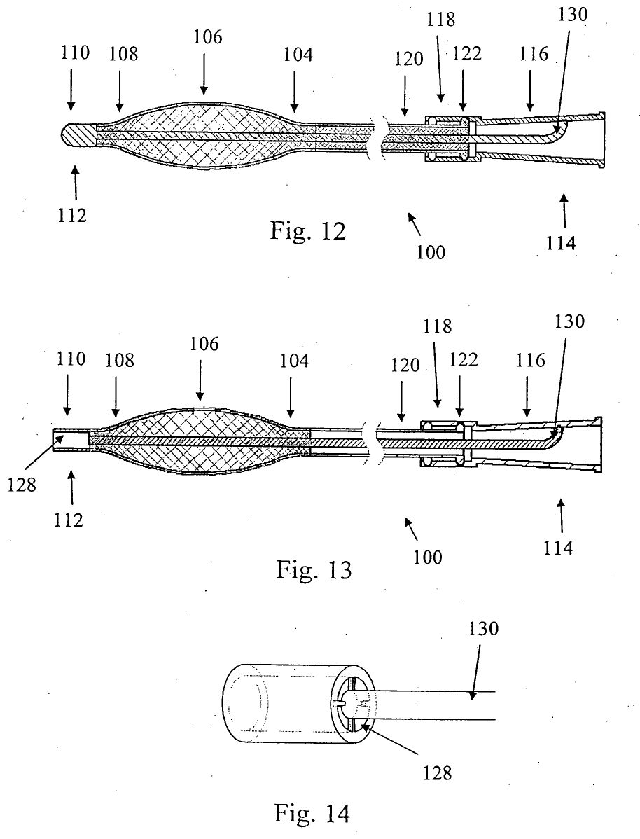

FIGS. 12 and 13 show two different embodiments of the medical device. The drainage section 120 in FIG. 12 comprises a braiding whereas the drainage section 120 in FIG. 13 does not comprise a braiding. The provision of a braiding reinforces the drainage section such that a conduit with a larger cross-section may be provided for the same outer diameter of the device. In FIG. 12 the tip is solid whereas the tip in FIG. 13 is ring shaped with an open end. The ring shaped tip is shown in detail in FIG. 14 wherein it may be seen that it a conduit 128 which makes is possible to provide a flow passage from the bladder into the drainage section 120 even if the tip 120 closely fits the drainage section 120. The ring shaped tip is attached to the deployment member 130.

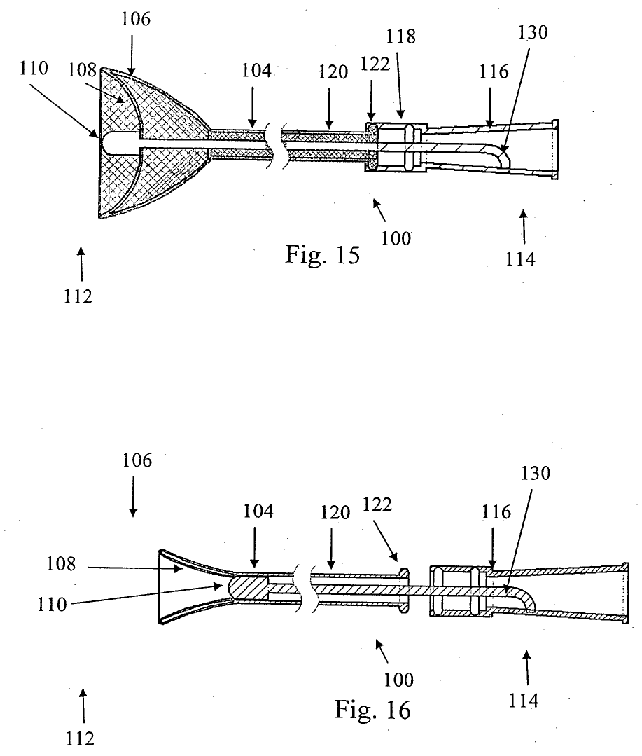

A deployment member 130 may be used to move the tip 110. The tip is gradually moved in FIG. 11-16 from a state wherein the braided retention section 102 is stretched in FIG. 11 in to a position wherein the braided retention section forms a funnel 132 in FIG. 15. In FIG. 15 the tip is moved to a position wherein it does not constitute the distal end of the medical device. On the contrary the distal end in FIG. 15 is the rim portion 134 of the funnel.

The connector 116 may be transparent such that the deployment member 130 is visible, and at the same time the deployment member 130 may be provided with sections of different colour indicating the position of the tip. Thus when the deployment member has been pulled out to a predetermined position a signalling colour e.g. red may be visible and indicate a relative position of the tip in relation to the conduit, the drainage section or the retention section. In another embodiment the deployment member comprises a visible scale which indicated the position of the tip.

In FIG. 16 may be seen that in some embodiments the connector 116 may be disconnected from the drainage section 120 in order to obtain a funnel shape of the retention section.

1.-14. (canceled)

15. A drainage catheter, comprising:
an elongated drainage section defining a lumen for draining fluid, said drainage lumen having an opening at a distal end;
a retention section having a configuration wider than said distal end of said drainage lumen and adapted to retain said catheter at a desired site, said retention section defining a lumen in communication with said drainage lumen and being located distally of said drainage section;
a tip having a first position located distally of said retention section, said tip being connected to said retention section when said retention section is in said wider configuration;
a deployment member fixedly connected to said tip and operable by a user to retract said tip from said first position to a second position wherein said tip is at least partially located in at least one of said retention section and said drainage section, wherein said tip in said second position does not block said drainage lumen so that fluid may flow through said drainage lumen; and
a lock adapted to lock said tip in at least one of said first position and said second position;
wherein both of said drainage section and said retention section are braided, and said braided retention section is a continuation of said braided drainage section.

16. The drainage catheter according to claim 15, further comprising an indicator which is adapted to indicate a position of said tip relative to at least one of said drainage section and said retention section.

17. The drainage catheter according to claim 16 wherein said indicator is adapted to indicate that said tip is a distal-most end of said catheter.

18. The drainage catheter according to claim 16, wherein said indicator is adapted to indicate that said tip is received within said retention section.

19. The drainage catheter according to claim 15 wherein said deployment member is connected to said retention section and is operable to change said retention section between a narrow configuration suitable for insertion of said catheter and said wider configuration.

20. The drainage catheter according to claim 19 wherein said tip is locatable in said first position when said retention section is in said narrow configuration, and said tip is locatable in said second position when said retention section is in said wider configuration

21. The drainage catheter of claim 16 wherein said indicator includes a tactile indicator.

22. The drainage catheter of claim 16 wherein said indicator includes a visual indicator.

23. The drainage catheter of claim 22 wherein said visual indicator includes an indicator provided on a proximal end of said deployment member.

24. The drainage catheter of claim 23 wherein said visual indicator on said proximal end of said deployment member includes a particular color.

25. The drainage catheter of claim 22 wherein a proximal portion of said catheter is transparent so that said visual indicator is observable through said transparent proximal portion.

26. The drainage catheter according to claim 15 wherein said lock is adapted to lock said deployment member so that said retention section remains in one of a narrow configuration suitable for insertion of said catheter into a patient and said wider configuration, and so that said tip correspondingly remains in one of said first position and said second position.

27. The drainage catheter of claim 15 including a connector for connecting said catheter to a device for collecting fluids drained through said catheter.

28. The drainage catheter of claim 16 wherein said indicator is adapted to indicate at least one of said first position and said second position.

29. The drainage catheter according to claim 15 wherein said braided drainage section includes filaments defining openings therebetween, and further including a polymer that blocks said openings.

30. A method of using a drainage catheter, comprising:
providing a drainage catheter having an elongated drainage section defining a lumen for draining fluid, the drainage lumen having an opening at a distal end, a retention section having a narrow configuration suitable for insertion into a patient and a wider configuration for retaining the catheter at a desired site, the retention section in the wider configuration defining a lumen in communication with the drainage lumen and being located distally of the drainage section, and a tip having a first position located distally of the retention section when the retention section is in the narrow configuration, the tip being moveable into a second position when the retention section is in the wider configuration wherein the tip is at least partially located within at least one of the retention section and the drainage section but does not block the drainage lumen so that fluid may flow through the drainage section lumen when the tip is in the second position, wherein both of the drainage section and the retention section are braided and the braided retention section is a continuation of the braided drainage section, and a lock adapted to lock said tip in at least one of said first position and said second position;
inserting the catheter into a patient with the tip in the first position and the retention section in the narrow configuration;
changing the retention section to the wider configuration;
retracting the tip from the first position to the second position; and
locking the tip in the second position.
